(19) United States
(12) Patent Application Publication
(10) Pub. No.: US 2025/0216659 A1
(43) Pub. Date: Jul. 3, 2025

(54) RAPID OPTICAL TISSUE SCANNER AND SCANNING METHOD

(71) Applicants: POSTECH RESEARCH AND BUSINESS DEVELOPMENT FOUNDATION, Pohang-si, Gyeongsangbuk-do (KR); GWANGJU INSTITUTE OF SCIENCE AND TECHNOLOGY, Gwangju (KR)

(72) Inventors: Ki Hean KIM, Pohang-si, Gyeongsangbuk-do (KR); Soo Hyun PARK, Pohang-si, Gyeongsangbuk-do (KR); Bum Ju KIM, Pohang-si, Gyeongsangbuk-do (KR); Eui Heon CHUNG, Gwangju (KR)

(73) Assignees: POSTECH RESEARCH AND BUSINESS DEVELOPMENT FOUNDATION, Pohang-si, Gyeongsangbuk-do (KR); GWANGJU INSTITUTE OF SCIENCE AND TECHNOLOGY, Gwangju (KR)

(21) Appl. No.: 18/852,461

(22) PCT Filed: Dec. 30, 2022
(86) PCT No.: PCT/KR2022/021687

§ 371 Date: Sep. 29, 2024

(30) Foreign Application Priority Data
Mar. 31, 2022 (KR) 10-2022-0040835
Dec. 29, 2022 (KR) 10-2022-0189457

(51) Int. Cl. G02B 21/00

(52) U.S. Cl. CPC G02B 21/0032; G02B 21/008

(57) ABSTRACT

A rapid optical tissue scanner includes: a light source unit for irradiating a sample with a light source beam; a cylindrical lens for transforming the light source beam into a light sheet; a sample mounting unit on which the sample is mounted; an objective lens disposed between the cylindrical lens and the sample mounting unit to irradiate the sample with the light source beam transformed into a light sheet; and a photographing unit for receiving an excitation beam, which is obtained by excitation from the sample, from the objective lens and photographing image information of the sample.

### CROSS REFERENCE TO RELATED APPLICATIONS

This application is a National Stage Application of PCT International Patent Application No. PCT/KR2022/021687 filed on Dec. 30, 2022, under 35 U.S.C. § 371, which claims priority to Korean Patent Application Nos. 10-2022-0040835 filed Mar. 31, 2022, and 10-2022-0189457 filed on Dec. 29, 2022, respectively, which are all hereby incorporated by reference in their entirety.

### NATIONAL RESEARCH AND DEVELOPMENT PROJECT SUPPORTING THE PRESENT DISCLOSURE

Project Serial Number: 1711138095

Project Number: RS-2020-KD000076

Ministry Name: the Ministry of Science and ICT, the Ministry of Trade, Industry and Energy, the Ministry of Health & Welfare, the Ministry of Food and Drug Safety

Project Management (Specialized) Institution: Korea Medical Device Development Fund

Research Business: Korea Medical Device Development Fund

Research Topic: Rapid high sensitivity optical tissue scanner and AI image processing

Contribution Ratio: 1/1

Project Performing Institution: Pohang University of Science & Technology

Research Period: From Mar. 1, 2021 to Feb. 28, 2022.

### TECHNICAL FIELD

The present disclosure relates to a rapid optical tissue scanner and scanning method. More particularly, it pertains to a rapid optical tissue scanner and scanning method capable of providing high-resolution and high-contrast cellular images, comparable to existing pathological tissue examination methods, while maintaining the speed advantages of the existing optical tissue examination methods.

### DESCRIPTION OF THE RELATED ART

Rapid optical tissue scanning is a high-speed, high-contrast, and high-resolution imaging method by utilizing a light sheet microscopy, and can be utilized as a high-speed optical tissue examination method that visualizes cellular structures on the surface of biological tissue collected surgically or by needle biopsy.

The existing optical tissue examination methods for rapid tissue examination have utilized examination methods based on confocal microscopy and two-photon microscopy, or light sheet microscopy (LSM).

However, there are problems in that confocal or two-photon microscopes have a slow photographing speed due to the point-by-point scanning method and the existing light sheet microscopy has a relatively low image resolution to increase an imaging area.

Therefore, there is a need for a rapid optical tissue scanner that can provide cell image quality with high resolution and high contrast at the level of the existing pathological tissue examination methods, while maintaining the speed of the existing optical tissue examination methods.

### Technical Problem

A task of the present disclosure to solve the described problem is to provide a rapid optical tissue scanner and scanning method capable of providing cell image quality with high resolution and high contrast at the level of the existing pathological tissue examination methods while maintaining the speed of the existing optical tissue examination methods.

A task to be solved by the present disclosure is not limited to the task mentioned above, and other tasks not mentioned will be clearly understood by those skilled in the art to which the present disclosure belongs from the following description.

### DOCUMENT OF RELATED ART

(Patent Document 1) Korean Patent Application Publication No. 10-2014-0126554 A (Invention title: Medical imaging scanner, published on Oct. 31, 2014)

### Technical Solution

To solve the described task, the present disclosure provides a rapid optical tissue scanner including a light source unit radiating a light source beam onto a sample, a cylindrical lens transforming the light source beam into a light sheet shape, a sample mounting unit where the sample is mounted, an objective lens that is disposed between the cylindrical lens and the sample mounting unit for irradiating the sample with the light source beam transformed into the light sheet shape, and a photographing unit that receives an emission beam generated in the sample from the objective lens and photographs image information of the sample.

In addition, the present disclosure provides a rapid optical tissue scanner including a light source unit radiating a light source beam onto a sample, a cylindrical lens transforming the light source beam into a light sheet shape, a sample mounting unit where the sample is mounted, a first objective lens that is disposed between the cylindrical lens and the sample mounting unit for irradiating the sample with the light source beam transformed into the light sheet shape, a second objective lens that is disposed perpendicular to the light source beam and obtains image information of the sample by receiving an emission beam generated in the sample, and a photographing unit that photographs image information of the sample.

Herein, a tunable focus unit disposed between the cylindrical lens and the sample mounting unit or between the light source unit and the cylindrical lens may be further included to axially translate a focus of the light sheet beam in the sample.

In addition, the tunable focus unit may include a deformable mirror.

In addition, the tunable focus unit may include a tunable lens.

In addition, the light source unit may include a first light source radiating a first light source beam of a first wavelength, a second radiating a second light source beam of a second wavelength, and a light integration dichroic mirror integrating the first light source beam and the second light source beam into the light source beam.

In addition, the present disclosure provides a rapid optical tissue scanning method that includes a light radiating step of radiating a light source beam onto a sample by a light source unit, a beam shape transforming step of transforming the light source beam into a light sheet shape by a cylindrical lens, a focus tuning step of tuning a focus of the light source beam focused on the sample by a tunable focus unit, and a photographing step of photographing image information of the sample by receiving an emission beam generated in the sample from an objective lens.

### Advantageous Effects

The rapid optical tissue scanner and scanning method according to the present disclosure have the following effects. First, there is an advantage of scanning and photographing the sample more quickly by radiating the light sheet onto the sample to be observed, exciting in a plane basis and obtaining a signal.

Second, there is an advantage of uniformly illuminating a photographing area with a tightly focused beam sheet and then obtaining high-resolution and high-contrast cell image quality by changing the axial position of the light sheet focus, where a sample is irradiated by the tightly focused sheet, by a tunable focus unit.

Third, there is an advantage of photographing all the cell nucleus and cytoplasm of the sample by individually obtaining a first image information generated from a first light source and a second image information generated from a second light source while simultaneously radiating a first light source beam and a second light source beam onto a sample to be observed and then obtaining image information from an emission beam generated in the sample.

Fourth, there is an advantage of identifying a flat image of a sample using a single objective lens without a separate image correction by changing the angle of an excitation beam and correcting a tilted image plane by an image tilt correction unit.

### BRIEF DESCRIPTION OF THE DRAWINGS

FIG. 1 is a view schematically showing a configuration for explaining a first exemplary embodiment of a rapid optical tissue scanner according to the present disclosure.

FIG. 2 is a view for explaining an image separation unit, which is a component of a rapid optical tissue scanner of FIG. 1.

FIG. 3 is a view schematically showing a configuration for explaining a second exemplary embodiment of a rapid optical tissue scanner according to the present disclosure.

FIG. 4 illustrates a view (a) for explaining an example of operation when a tunable focus unit configured in a first exemplary embodiment and a second exemplary embodiment of a rapid optical tissue scanner according to the present disclosure is configured as a tunable lens, and a view (b) for explaining an example of operation when a tunable focus unit configured in a first exemplary embodiment and a second exemplary embodiment of a rapid optical tissue scanner according to the present disclosure is configured as a deformable mirror.

FIG. 5 illustrates a view (a) showing a photograph of human pancreatic tissue photographed at a 0 μm deep from the surface by a rapid optical tissue scanner according to the present disclosure, a view (b) showing a photograph of human pancreatic tissue photographed at a 70 μm deep from the surface by a rapid optical tissue scanner according to the present disclosure, and a view (c) showing a photograph of human pancreatic tissue photographed by changing the focus depth ranging of 0 μm to 140 μm from the surface by a rapid optical tissue scanner according to the present disclosure.

FIG. 6 illustrates a view (a) showing a photograph of a cross-section of human colon tissue stained with two fluorescent dyes by a rapid optical tissue scanner according to the present disclosure, and a view (b) showing a photograph of a cross-section of human colon tissue stained with hematoxylin and eosin in pseudo by a rapid optical tissue scanner according to the present disclosure.

FIG. 7 is a view showing steps of a rapid optical tissue scanning method according to the present disclosure.

### DETAILED DESCRIPTION OF THE INVENTION

The advantages and features of the present disclosure and methods for achieving them will become apparent with reference to the exemplary embodiments described in detail below together with the accompanying drawings. However, the present disclosure may be implemented in various different forms rather than limited to the exemplary embodiments disclosed below, and the exemplary embodiments may be provided only to make the present disclosure complete and to fully inform those skilled in the art to which the present disclosure belongs of the scope of the present disclosure, which is only defined by the scope of the claims. Throughout the specification, the same reference numeral may refer to the same component.

Terms used in the present specification may be used to describe specific exemplary embodiments and may not be intended to limit the present disclosure. As used in the present specification, a singular form may include a plurality of forms unless the context clearly indicates otherwise. In addition, when it is said that a part throughout the present specification “includes” a component, it may mean that other components may be further included unless the context specifically indicates the contrary.

When it is said that a component is “connected” or “linked” to another component, it should be understood that it may be directly connected or linked to that other component, but that there may be other components in between. Meanwhile, when it is said that a component is “directly connected” or “directly linked” to another component, it should be understood that there are no other components in between. Other expressions intended to describe the relationship between components should be interpreted similarly.

All terms, including technical or scientific terms used in the present specification, may have the same meaning as commonly understood by those skilled in the art to which the present disclosure belongs, unless otherwise defined. Terms such as those defined in commonly used dictionaries should be construed as having a meaning consistent with the meaning of the context of the relevant technology and are not interpreted to have an ideal or overly formal meaning unless explicitly defined in the present specification.

Terms such as one surface, the other surface, the upper end, the lower end, the upper surface, the lower surface, the upper part, the lower part and the like used in the present specification may be used to distinguish the relative positions of components.

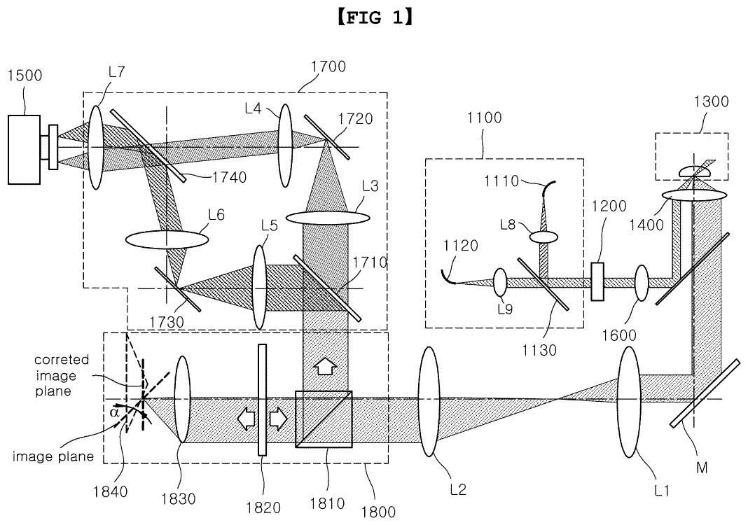

A rapid optical tissue scanner according to the present disclosure will be described with reference to FIGS. 1 to 6.

First, FIG. 1 is a view schematically showing a configuration for explaining a first exemplary embodiment of a rapid optical tissue scanner according to the present disclosure, and the first exemplary embodiment of the rapid optical tissue scanner according to the present disclosure may include a light source unit 1100, a cylindrical lens 1200, a tunable focus unit 1600, an objective lens 1400, an image tilt correction unit 1800, a sample mounting unit 1300, an image splitter unit 1700, a photographing unit 1500, a lens group and a controller (not shown).

The light source unit 1100 may radiate a light source beam onto a tissue cell sample which is a target to be observed and photographed by the rapid optical tissue scanner according to the present disclosure.

Specifically, the light source unit 1100 may include a first light source 1110, a second light source 1120, and a dichroic mirror 1130.

The first light source 1110 may radiate a first light source 1110 beam of a first wavelength onto the sample, and it may be preferable that the first light source 1110 beam is a wavelength at which DAPI stained on the cell nucleus of the sample is fluorescently excited.

The second light source 1120 may radiate a second light source 1120 beam of a second wavelength onto the sample, and it may be preferable that the second light source 1120 beam is a wavelength at which ATTO 655 NHS esters stained on the cytoplasm of the sample are fluorescently excited.

The light integration dichroic mirror 1130 may integrate the first light source 1110 beam and the second light source 1120 beam into the light source beam so that the first light source 1110 beam and the second light source 1120 beam can simultaneously irradiate the sample in the same area and focus.

The cylindrical lens 1200 may allow the light source beam to excite the sample in a plane basis by transforming the light source beam radiated from the light source unit 1100 into a light sheet shape.

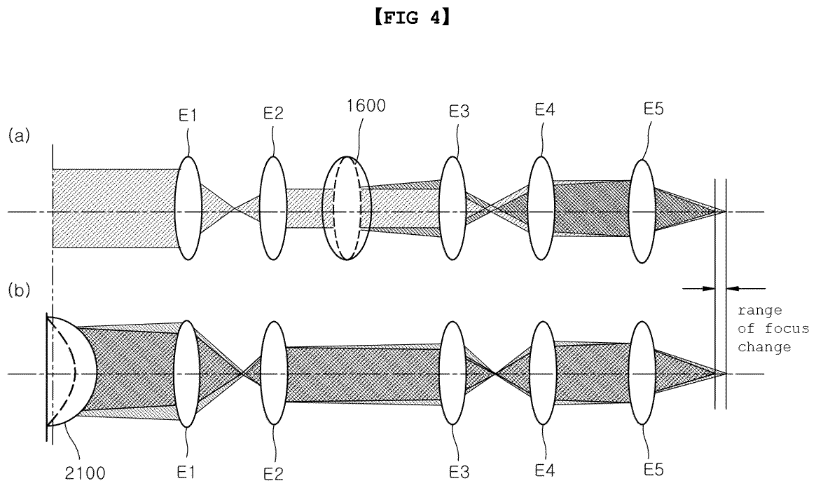

The tunable focus unit 1600 may expand a scan area in the sample by axially changing the focus position of the light sheet in the sample, and a detailed description of the tunable focus unit 1600 will be described later with reference to FIG. 4.

The objective lens 1400 may be disposed between the cylindrical lens 1200 and the sample mounting unit 1300 so that the light source beam transformed into a light sheet shape can focus on the sample and simultaneously the emission beam excited in the sample by the light source beam can move.

That is, the objective lens 1400 may be configured as a single unit, the excitation beam may pass through a partial aperture area of the objective lens 1400, and the excitation beam may move passing through a partial aperture area of the objective lens 1400 in the first exemplary embodiment of the rapid optical tissue scanner according to the present disclosure.

The image tilt correction unit 1800 may be disposed between the objective lens 1400 and the photographing unit 1500 and may change the angle of the tilted image plane.

That is, the excitation beam may be focused on a tilted plane in the sample using a portion of the objective lens 1400 and emission light may be generated in the tilted illumination plane in the sample and collected by the objective lens 1400 for image information. The tilted image plane in the sample may need to be rotated.

Accordingly, a process of rotating the tilted image plane may be performed using the image tilt correction unit 1800. After the rotation, emission light from the un-tilted image plane may be photographed by the photographing unit 1500.

Specifically, the image tilt correction unit 1800 may include a polarized beam splitter (PBS) 1810, a quarter wave plate (QWP) 1820, a relay objective lens 1830 and a prism mirror 1840.

The emission beam excited in the sample may reach the image tilt correction unit 1800 by passing through the polarized beam splitter 1810, the QWP 1820, and the relay objective lens 1830 in the image tilt correction unit 1800 may generate another tilted image plane. The tilt angle of the new image plane may be determined by the magnification of the image relay and refractive indices of the immersion media used for the objective lenses.

At this time, the prism mirror 1840 may be positioned in the focus of the relay objective lens 1830 and oriented at half the tilt angle, and the image plane formed by reflected light on the prism mirror 1840 may be rotated to the focal plane of the relay objective lens 1830.

The emission light, which is reflected on the tilted prism mirror 1840 and collected by the relay objective lens 1830, may pass through the QWP 1820 and then be reflected on the polarization beam splitter 1810 toward the image splitter unit 1700 and the photographing unit 1500.

The sample mounting unit 1300 may be mounted with the sample, wherein the cell nucleus of the sample is stained with DAPI and the cytoplasm of the sample is stained with ATTP 655 NHS ester.

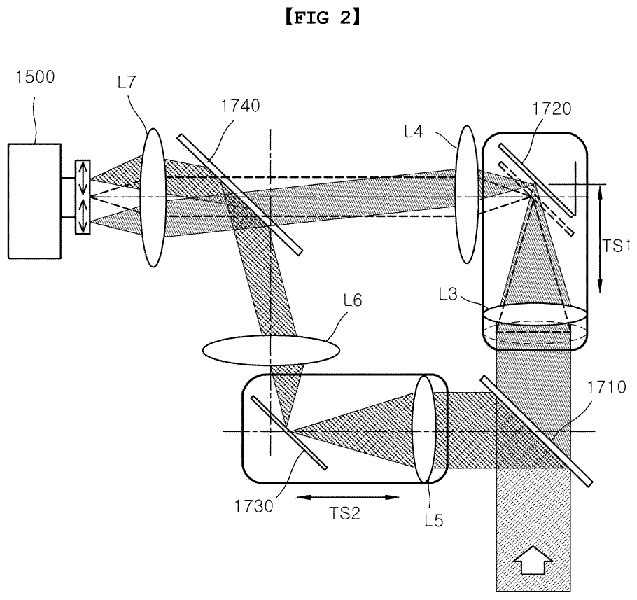

The image splitter unit 1700 may separate the emission light from the sample after rotation of the tilted image plane and form two images of the sample: the first image information generated from the first light source 1110 and the second image information generated from the second light source 1120, and a detailed description of the image splitter unit 1700 will be described later with reference to FIG. 2.

The photographing unit 1500 may photograph the image information of the sample.

The lens group may include a first lens L1 to ninth lens L9 (hereinafter, the lens), and the lens may be disposed in an area between the light source unit 1100, the cylindrical lens 1200, the tunable focus unit 1600, the objective lens 1400, the sample mounting unit 1300, the image splitter unit 1700 and the photographing unit 1500, and may perform a role of magnification, focusing, etc. of the light source beam and the excitation beam.

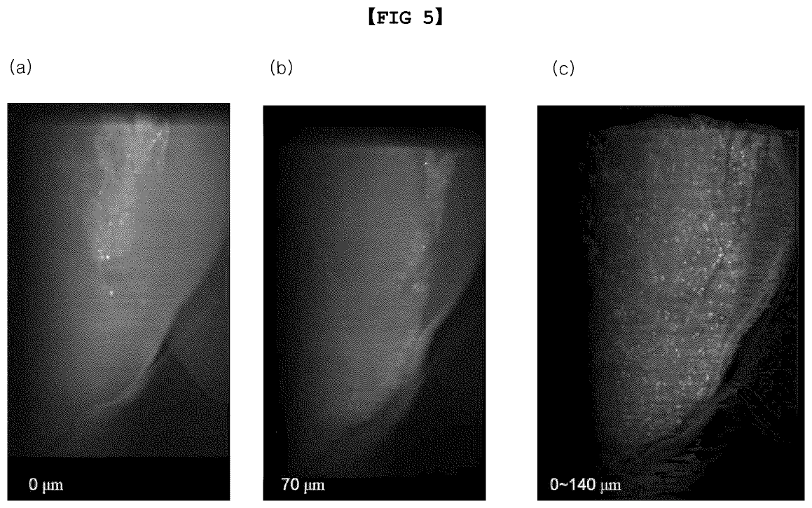

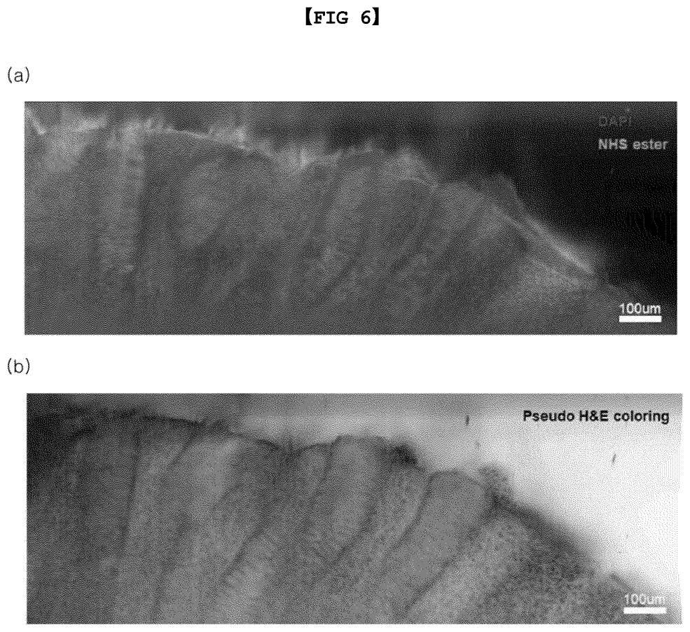

The controller may perform an extended depth of field image processing and a pseudo hematoxylin & eosin H&E coloring, which will be described in detail later with reference to FIGS. 5 and 6.

FIG. 2 is a view for explaining the image separation unit 1700, which is a component of a rapid optical tissue scanner of FIG. 1. The image splitter unit 1700 may include an image separation dichroic mirror 1710, a first mirror 1720, a second mirror 1730, a third lens L3 to a seventh lens L7, an image integration dichroic mirror 1740, a first transfer device TS1, and a second transfer device TS2, which are disposed in a-shape structure.

The image separation dichroic mirror 1710 may separate the emission beam from the untilted image plane, which is generated by the image tilt correction unit 1800.

That is, the first image information generated by the first light source 1110 beam and the second image information generated by the second light source 1120 beam may be separated by the image separation dichroic mirror 1710.

Thereafter, the first image information may pass through the third lens L3, reflect on the first mirror 1720, pass through the fourth lens L4 in the form of collimated beam. The collimated first emission beam may then pass through the integration dichroic mirror 1740. The second image information may pass through the fifth lens L5, reflect on the second mirror 1730, pass through the sixth lens L6 in the form of collimated beam. The collimated second emission beam may then reflect on the integration dichroic mirror 1740.

The first transfer device TS1 may change the position of the first image information reaching the image integration dichroic mirror 1740 by by axially translating (pushing) the third lens L3 and the first mirror 1720, and the second transfer device TS2 may change the angle of the second emission beam reaching the image integration dichroic mirror 1740 in the opposite direction of the first emission beam by axially translating (pulling) the fifth lens L5 and the second mirror 1730. The angles of the first and second emission beams are in opposite directions.

That is, the first image information and the second image information may be focused in the two different areas of the photographing unit 1500 by reaching the image integration dichroic mirror 1740 at positive and negative angles, respectively. The photographing unit 1500 can simultaneously photograph the first image information and the second image information.

In this case, the transfer direction and distance of the first transfer device TS1 and the second transfer device TS2 may be determined according to the focal length ratio of the third lens L3 to the seventh lens L7.

The image integration dichroic mirror 1740 may form an integrated image information such that the photographing unit 1500 can integratedly photograph the first image information and the second image information.

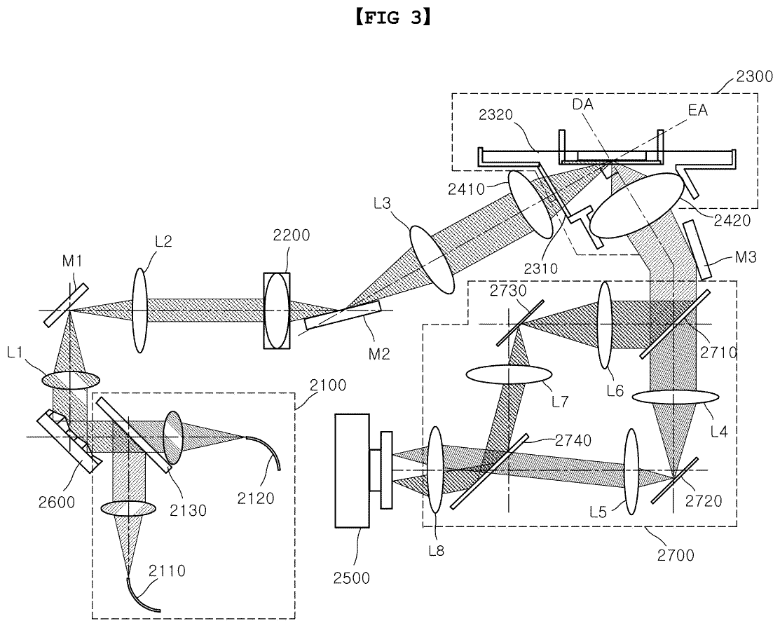

FIG. 3 is a view schematically showing a configuration for explaining a second exemplary embodiment of a rapid optical tissue scanner according to the present disclosure, and the second exemplary embodiment of the rapid optical tissue scanner according to the present disclosure may include a light source unit 2100, a tunable focus unit 2600, a cylindrical lens 2200, a first objective lens 2410, a second objective lens 2420, a sample mounting unit 2300, an image splitter unit 2700, a photographing unit 2500, a lens group and a controller (not shown).

The light source unit 2100 may radiate a light source beam onto the sample.

The tunable focus unit 2600 may tune a focal plane where the light source beam is focused on the sample, and the detailed description of the tunable focus unit 2600 will be described later with reference to FIG. 4.

The cylindrical lens 2200 may transform the light source beam into a light sheet shape.

The sample mounting unit 2300 may include a window 2310, which is positioned perpendicular to the excitation axis (EA) of the first objective lens 2410, and a liquid prism 2320, and when optical aberration caused by the the sample is mounted, an excitation beam incident at a specific angle with the sample may be minimized.

The first objective lens 2410 may be disposed between the cylindrical lens 2200 and the sample mounting unit 2300 and may radiate the light source beam transformed into the light sheet shape onto the sample.

At this time, the first objective lens 2410 may be an objective lens with a relatively low numerical aperture and a long working distance, and may excite in the sample with a focus of the light sheet shape while forming a specific angle with the sample.

The second objective lens 2420 may obtain the image information of the sample by receiving the emission light generated in the sample, and may obtain the image information from the emission light while being immersed in the liquid prism 2320 and being positioned orthogonal to the excitation light sheet.

The image separation splitter unit 2700 may split the emission light spectrally and generate two separate image information of the sample: the first image information generated from the first light source 2110 and the second image information generated from the second light source 2120.

The photographing unit 2500 may photograph the two image information of the sample simultaneously.

The lens group may include the first lens L1 to the eighth lens L8 and may perform a role of magnification, focusing, etc. of the light source beam and the excitation beam.

In addition, the mirror groups M1, M2, M3 may reflect the light source beam and the emission beam to change their directions.

In addition, the detailed description of the light source unit 2100, the cylindrical lens 2200, the image splitter unit 2700, the photographing unit 2500, and the lens group may be the same as the description of the first exemplary embodiment of the rapid optical tissue scanner according to the present disclosure, and thus the description thereof will be omitted.

FIG. 4a is a view for explaining an example of operation when tunable focus units 1600, 2600 configured in a first exemplary embodiment and a second exemplary embodiment of a rapid optical tissue scanner according to the present disclosure is configured as a tunable lens.

FIG. 4b is a view for explaining an example of operation when a tunable focus unit 1600, 2600 configured in a first exemplary embodiment and a second exemplary embodiment of a rapid optical tissue scanner according to the present disclosure is configured as a deformable mirror.

As described above, the tunable focus units 1600, 2600 may be disposed between the cylindrical lenses 1200, 2200 and the sample mounting units 1300, 2300 or between the light source units 1100, 2100 and the cylindrical lenses 1200, 2200 and may tune the focus position of the light source beam axially in the sample.

In this case, the tunable focus units 1600, 2600 may be composed of a deformable mirror as shown in FIG. 4a, or a tunable lens as shown in FIG. 4b.

That is, being selectively configured according to the location, where the tunable focus units 1600, 2600 are disposed, and the travel route of light, there is an advantage of uniformly illuminating a photographing area with a tightly focused light sheet and then obtaining high-resolution and high-contrast cell image quality by allowing the tunable focus units 1600, 2600 to to axially change the focal position of the illumination light in the sample.

Referring to FIG. 5, the extended depth of field image processing of the rapid optical tissue scanner according to the present disclosure will be explained with reference to a photograph of human pancreatic tissue as follows.

FIG. 5a is a view showing a photograph of human pancreatic tissue photographed with the illumination light sheet focused at 0 μm deep from the sample surface by a rapid optical tissue scanner according to the present disclosure, FIG. 5b is a view showing a photograph of human pancreatic tissue photographed with the illumination light sheet focused at 70 μm deep from the surface by a rapid optical tissue scanner according to the present disclosure, and FIG. 5c is a view showing a photograph of human pancreatic tissue photographed with the axial scanning of the illumination light sheet focus of 0 μm to 140 μm deep from the surface by a rapid optical tissue scanner according to the present disclosure.

That is, as shown in FIGS. 5a and 5b, the images are acquired with fixed focal depths of 0 μm and 70 μm from the surface and in-focus information is identified only in narrow depth ranges.

By performing the extended depth of field image processing in the controller, the image information, which is scanned in a plane basis of including a volume information of the three-dimensional in-focus and out-of-focus areas photographed by the photographing unit 1500 may be converted into an en-face surface image by synthesizing successive local focus images. In this case, the extended depth of field image processing may use the F-stack algorithm (open-source, Joe Yeh, MIT license).

As a result, by performing the extended depth of field image processing in the controller, it may be possible to visualize the depth range information of 140 μm in focus as a single image by collecting images with the axial scanning of the tightly focused illumination light sheet.

Referring to FIG. 6, the human colon tissue photographed by the rapid optical tissue scanner according to the present disclosure will be described as follows.

FIG. 6a is a view showing a photograph of a cross-section of human colon tissue stained with two fluorescent dyes by a rapid optical tissue scanner according to the present disclosure, and FIG. 6b is a view showing a photograph of a cross-section of human colon tissue stained with hematoxylin and eosin in pseudo by a rapid optical tissue scanner according to the present disclosure.

In the case of FIG. 6a, DAPI stained on the cell nucleus of the sample may be red, and NHS ester stained on the cell nucleus of the sample may be green such that the cell nucleus and cytoplasm can be clearly identified.

In the case of FIG. 6b, by performing the pseudo hematoxylin and eosin coloring in the controller, it may be possible to identify an image similar to that of the existing tissue biopsy that examines human colon tissue.

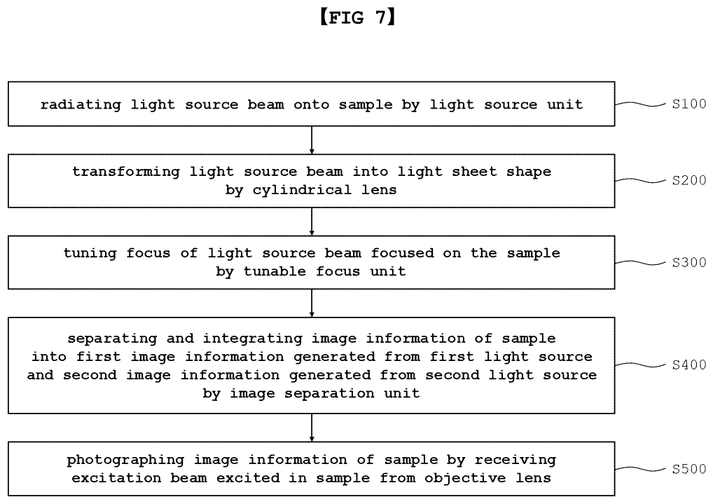

FIG. 7 is a view showing steps of a rapid optical tissue scanning method according to the present disclosure.

According to FIG. 7, the rapid optical tissue scanning method according to the present disclosure may include a light radiating step S100, a beam shape transforming step S200, a focus tuning step S300, an image separation/integration step S400, and a photographing step S500.

In the light radiating step S100, the light source units 1100, 2100 may radiate the light source beam onto the sample, and in the beam shape transforming step S200, the cylindrical lenses 1200, 2200 may transform the light source beam into the light sheet shape.

In the focus tuning step S300, the tunable focus units 1600, 2600 may tune change the focus location of the light sheet in the sample, and in the image separation/integration step S400, the image splitter units 1700, 2700 may separate and integrate the image information of the sample into the first image information generated from the first light sources 1110, 2110 and the second image information generated from the second light sources 1120, 2120.

In the photographing step S500, the photographing units 1500, 2500 may receive the emission beam generated in the sample from the objective lens 1400 (or a second objective lens 2420) and may photograph the image information of the sample.

In addition, a detailed description of the rapid optical tissue scanning method according to the present disclosure may correspond to the first exemplary embodiment and the second exemplary embodiment of the rapid optical tissue scanner according to the present disclosure described above, and thus a description thereof will be omitted.

Although the preferred exemplary embodiments of the present disclosure have been illustrated and described with reference to the drawings, the present disclosure may be not limited to the specific exemplary embodiment described above, and various modifications may be performed by those of ordinary skill in the technical field to which the present disclosure belongs without departing from the gist of the present disclosure claimed in the claims, and these modified exemplary embodiments should not be individually understood from the technical idea or prospect of the present disclosure.

### INDUSTRIAL APPLICABILITY

It can be applied to a high-speed optical tissue examination device, a microscope, a scanner or the like which visualizes cell structures on the surface of biological tissue.

1. A rapid optical tissue scanner, the scanner comprising:
a light source unit radiating a light source beam onto a sample;
a cylindrical lens transforming the light source beam into a light sheet shape;
a sample mounting unit where the sample is mounted;
an objective lens that is disposed between the cylindrical lens and the sample mounting unit for both irradiating the sample with the light source beam transformed into the light sheet and for collecting emission light from the sample; and
a photographing unit that receives an emission beam generated in the sample from the objective lens and photographs two image information of the sample simultaneously.

2. A rapid optical tissue scanner, the scanner comprising:
a light source unit radiating a light source beam onto a sample;
a cylindrical lens transforming the light source beam into a light sheet shape;
a sample mounting unit where the sample is mounted;
a first objective lens that is disposed between the cylindrical lens and the sample mounting unit for irradiating the sample with the light source beam transformed into the light sheet shape;
a second objective lens that is disposed perpendicular to the light source beam and obtains image information of the sample by receiving an emission beam generated in the sample; and
a photographing unit that photographs two image information of the sample simultaneously.

3. The scanner of claim 1, further comprising: a tunable focus unit that is disposed between the cylindrical lens and the sample mounting unit or between the light source unit and the cylindrical lens and axially scans a focus of the light sheet the sample.

4. The scanner of claim 3, wherein the tunable focus unit comprises a deformable mirror.

5. The scanner of claim 3, wherein the tunable focus unit comprises a tunable lens.

6. The scanner of claim 1, wherein the light source unit comprises:
a first light source radiating a first light source beam of a first wavelength;
a second light source radiating a second light source beam of a second wavelength; and
a light integration dichroic mirror integrating the first light source beam and the second light source beam into the light source beam.

7. A rapid optical tissue scanning method, the method comprising:
a light radiating step of radiating a light source beam onto a sample by a light source unit;
a beam shape transforming step of transforming the light source beam into a light sheet shape by a cylindrical lens;
a focus tuning step of tuning a focus of the light source beam focused on the sample by a tunable focus unit; and
a photographing step of photographing image information of the sample by receiving an emission beam generated in the sample from an objective lens.

8. The scanner of claim 2, further comprising: a tunable focus unit that is disposed between the cylindrical lens and the sample mounting unit or between the light source unit and the cylindrical lens and axially scans a focus of the light sheet the sample.

9. The scanner of claim 8, wherein the tunable focus unit comprises a deformable mirror.

10. The scanner of claim 8, wherein the tunable focus unit comprises a tunable lens.

11. The scanner of claim 2, wherein the light source unit comprises:
a first light source radiating a first light source beam of a first wavelength;
a second light source radiating a second light source beam of a second wavelength; and
a light integration dichroic mirror integrating the first light source beam and the second light source beam into the light source beam.
